(19) United States
(12) Patent Application Publication
(10) Pub. No.: US 2002/0019657 A1
(43) Pub. Date: Feb. 14, 2002

(54) Foot/ankle conformal therapy component of an animate body heat exchanger

(76) Inventor: William Elkins, San Jose, CA (US)

Correspondence Address: Michael B. Einschlag, Esq., ROSENLAW.COM, Suite 301, 702 Marshall St., Redwood City, CA, 94063, US

(21) Appl. No.: 09/771,124

(22) Filed: Jan. 26, 2001

Related U.S. Application Data
(60) Provisional application No. 60/178,983, filed on Jan. 28, 2000.

(51) Int. Cl. A61F 7/00

(52) U.S. Cl. 607/111; 607/108; 602/14

(57) ABSTRACT

A conformal therapy component of an animate body heat exchanger is described. The therapy component is designed for the foot and ankle of a wearer and includes both calf and vamp parts. A packet containing a thermally conductive gel is positioned at a gap between the vamp and calf parts to transfer the thermal energy state and the air pressure at the remainder of the component to the portion the wearer's foot under the gap. A pressure reactor sole also is provided to inhibit the pressure applied to the foot from tending to “flatten out” the foot.

### CROSS REFERENCE TO RELATED APPLICATIONS

Some of the subject matter described and claimed in this patent application is also disclosed in provisional Patent Application Ser. No. 60/178,983 filed Jan. 28, 2000, the priority of which is hereby claimed.

### DISCLOSURE

### BACKGROUND OF THE INVENTION

The present invention relates to temperature control apparatus for portions of a human or other animate body and, more particularly, to a conformal therapy component of an animate body exchanger, which component transfers a thermal energy state defined by the heat exchanger to a part of an animate body not in direct thermal conductive relationship the heat exchanger. The conformal therapy component of the invention also alleviates the effect of pressure applied by the therapy component to the wearer's body portion.

It is now common to apply cold and compression to a traumatized area of a human body to facilitate healing and to prevent unwanted consequences of the trauma. In fact, the acronym RICE (Rice, Ice, Compression, and Elevation) is now used by many.

Cold packing with ice bags or the like has traditionally been used to provide deep core cooling of a body part. Elastic wraps are often applied to provide compression.

It will be appreciated that the traditional techniques are quite uncontrollable. For example, the temperature of an ice pack will, of course, change when the ice melts; and it has been shown that the application of elastic wraps and, consequently, the pressure provided by the same, varies considerably even when the wrapping is done by an experienced individual.

Because of these and other difficulties, many in the field have turned to more complicated animate body heat exchangers. Most effective animate body heat exchangers have two major components—an external conformal therapy component for covering a body part to be subjected to heat exchange and compression, and a control component for producing a flowing heat exchange fluid and a pressurized gas. The external conformal therapy component typically includes two bladders, a first one of which confines a heat exchange liquid or other medium and a second of which overlays the first and confines gas pressure to be applied to the body part to inhibit edema and to apply pressure against the heat exchange liquid bladder to press it toward the body part.

In some instances it is impossible or impractical to design a conformal therapy component for an animate body heat exchanger which covers all of a body portion with which it is desired to provide a thermally conductive relationship. For example, in a foot/ankle therapy component there generally is a gap between the vamp and calf parts of the same, which gap is at the ankle of a wearer. It is desirable to provide both temperature control and pressure at such gap. Also, in some situations it is impossible or impractical to provide a therapy component which applies pressure equally to all parts of a body portion. In this connection, a foot/ankle therapy component typically is designed to apply pressure to the upper surface of the arch of a foot of a wearer but not to the sole. This unequal pressure can result in problems.

### SUMMARY OF THE INVENTION

A conformal therapy component is provided in accordance with the invention having features which overcome the above difficulties. It includes a packet containing a thermally conductive gel, which packet is positionable to be in thermally conductive relationship with a heat exchange fluid bladder to transfer a thermal energy state received from such bladder to a part of an animate body which is not in thermally conductive relationship with such bladder. It also includes a pressure reactor in the form of a closed compliant bladder which is positionable to alleviate the distortive effect of pressure applied by the remainder of the therapy component on a body portion.

The preferred therapy component of the invention is a foot/ankle therapy component. It includes both vamp and calf parts which when fitted about corresponding parts of a human foot/ankle leave a gap at a joint between the parts, i.e., a gap designed to be at the ankle of the wearer. In such an arrangement, the packet is in the form of a generally horizontal tongue which contains a thermally active gel and is designed to bridge such gap and transfer any thermal energy state and pressure received by it from the remainder of the splint to that part of the wearers foot not in thermal conductive and pressure relationship with the usual bladder provided for confining a heat exchange fluid. The pressure reliever (reactor) in such an arrangement is a sole designed to be positioned opposed to that part of the component which applies pressure—in the preferred embodiment at the sole of a wearer's foot opposed to the top of the foot adjacent the arch.

Most desirably, the foot/ankle therapy component of the preferred embodiment of the invention is, in essence, a boot having a covering made from a loop fastener material, and both the calf and vamp parts of the component include flaps which have hook fastening material to interact with the loop fastening surface of the vamp and calf parts to provide fastening in the common way typified by “Velcro” tape, to snugly fit about the foot/ankle and calf of a wearer.

Other features and advantages of the invention either will become apparent or will be described in connection with the following, more detailed description of a preferred embodiment of the invention and variations.

### BRIEF DESCRIPTION OF THE DRAWING

With reference to the attached sheets of drawing:

FIG. 1 is a partial perspective view showing a preferred foot/ankle embodiment of the invention fitted about the foot and ankle of a human user;

FIG. 2 is a side view of the preferred foot/ankle therapy component of the preferred embodiment;

FIG. 3 is an exploded, isometric view of the foot/ankle component incorporating the preferred embodiment of the invention;

FIG. 4 is an elevation and flattened out view showing the interior of the liquid flow bladder of the preferred embodiment;

FIG. 5 is an enlarged elevation view of a gel packet included as part of the combination of the preferred foot/ankle embodiment of the instant invention;

FIG. 6 is an enlarged, partial sectional view of the gel packet of FIG. 5, showing the packet's exterior walls confining a gel;

FIG. 7 is an enlarged, partial and broken away view somewhat like FIG. 2 illustrating details of the foot/ankle embodiment of the present invention; and

FIG. 8 is a partial sectional view somewhat similar to the view of FIG. 6 showing the external walls and the contained gel in the compliant sole of the preferred embodiment.

### DETAILED DESCRIPTION OF THE PREFERRED EMBODIMENT

The following, relatively detailed description is provided to satisfy the patent statutes. It will be appreciated by those skilled in the art, though, that various changes and modifications can be made without departing from the invention.

An animate body heat exchanger for a human is generally referred to in the drawings by the reference numeral 11. It includes a foot/ankle conformal therapy component 12 connected with a control unit 13, which control unit supplies the therapy component with both a flowing temperature controlled liquid and air pressure through suitable tubes which are covered by a tubular cord 14.

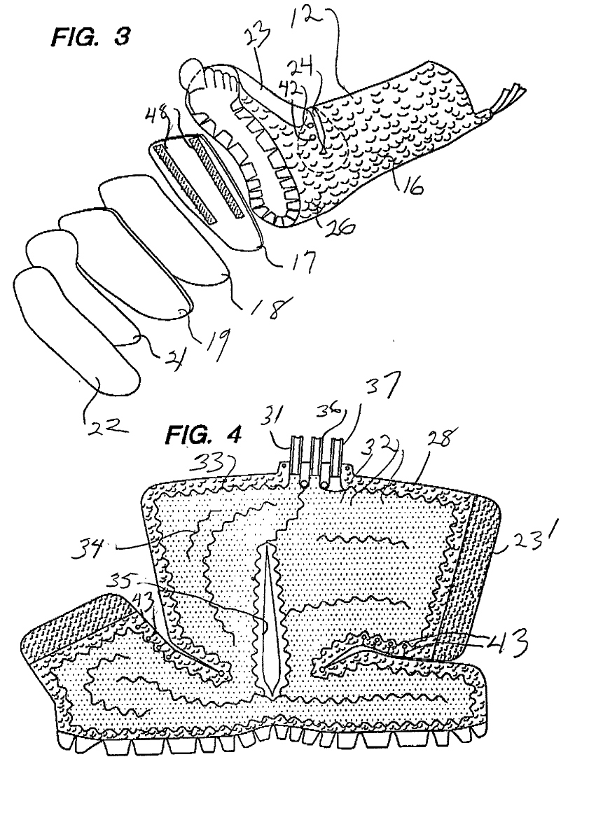

As best illustrated in FIGS. 2 and 3, the foot/ankle splint includes an outer foot covering 16, an inner gel sole 17 (to be described in more detail hereinafter), an inner sole 18 having a loop fastener surface facing the inner gel sole to interact therewith as will be described hereinafter, a formed rigid sole 19, a spacer 21 whose function will be described hereinafter, and a exterior high friction rigid sole 22. (It should be noted that all or some of the above parts, such as the spacer 21 and the outer rigid sole 22, could be part of a molded or cut sole similar to those used in athletic shoes at the time of filing the application.)

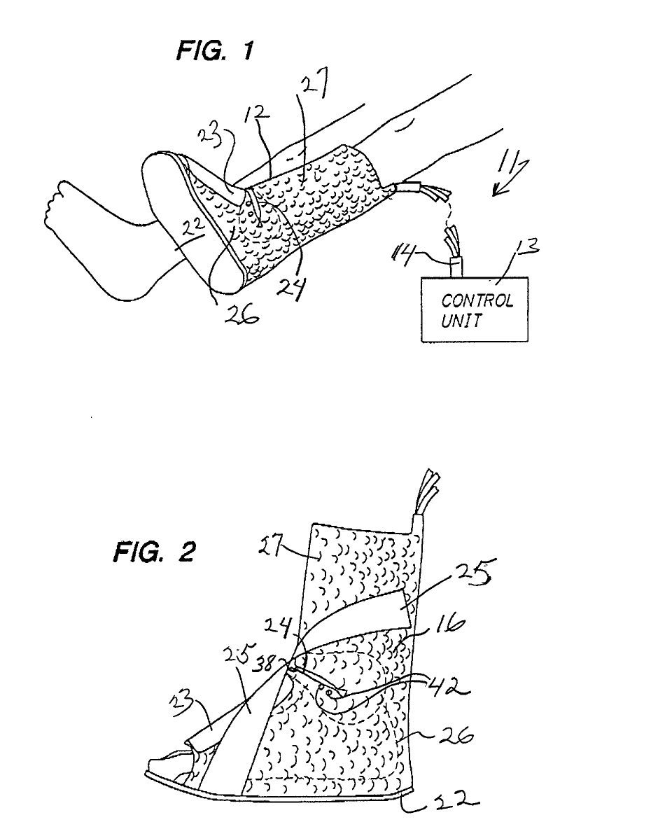

The foot/ankle therapy component further includes a pair of flaps 23 and 23′ of hook material for engaging the cover to close it, and a pair of straps 25 (not shown in FIGS. 1 and 3) which are suitably secured to the sides of the covering 16, such as by having ends sewn (not shown) to the outer covering adjacent the sole construction to extend up and across a gap 24 formed at the ankle of a wearer between the vamp 26 and calf portions 27 of the component.

As is illustrated, the therapy component is divided into two joined parts, the part defining the vamp and the part defining the calf. Each of the straps 23 is covered with a hook material to interactingly engage the loop material of its associated boot part. It should be noted that while as described the therapy component covering provides the loop material, from the broad standpoint it could be just the opposite, i.e., the covering providing the hook material and the flaps providing the loop material. Moreover, if desired, a separate supplementary collar section (not shown) can be provided for, for example, the vamp portion to enable the same to encircle wider calves than that for which it is designed. Such a supplemental collar will include, of course, both a loop material to interact with the flap 23′ and a hook portion to take the place of the flap.

Covering 16 covers a pair of overlapping bladders 28 and 29 (FIG. 7). Bladder 28 is for the purpose of confining a heat exchange fluid, preferably a liquid made up, for example, of a 20 percent propylene glycol solution of distilled or ionized water, having small amounts of a wetting agent to break surface tension and an antifungicide such as iodine.)

Bladder 28 is configured to be positioned in thermally conductive relationship with the foot/ankle of a human user. FIG. 4 shows the layout for such a bladder in more detail. It includes an entrance tube 31 which directs a liquid into the interior of the same. As illustrated, the interior includes a pattern of dots 32 or, in other words, a dot matrix to disperse the liquid. Preferably, such pattern conforms to that described in U.S. Patent Application Ser. No. 09/127,256 originally filed Jul. 31, 1998 and naming the instant inventor as the inventor.

The interior of bladder 28 is defined by a curvilinear border 33; and fences or, in other words, dividers 34 are provided to help direct the flow of liquid. In accordance with Patent Application Ser. No. 09/173,637 filed originally Jul. 21, 1998 and naming the inventor hereof as a co-inventor, the border 33 and the fences 34 are formed by a plurality of curvilinear ripples which generally are significantly shorter than the length of the border or fence, respectively, of which each is a part. This curvilinear ripple construction, among other things, prevents eddies from forming during liquid flow through the bladder. The layout includes a slit 35 which enables the component to conform to a wearer's foot when such component is appropriately shaped. (The heel of most wearers will extend beyond the ankle.)

The temperature controlled liquid flows through the bladder from inlet 31 to outlet 36 along paths defined by the fences 34. It is dispersed for flow throughout the full bladder by the matrix of dots 32.

The gas pressure bladder 29 overlays the fluid bladder 28 and presses the same against the foot/ankle being treated. Such gas pressure bladder is configured and positioned to press the heat exchange bladder against the foot and calf portion of the human. To these ends, the general configuration of the gas bladder is the same as that of the liquid bladder, and such bladder is supplied by the control unit 13 with air under pressure through inlet tube 37. The dot connections 32 are not included in such bladder, but the fences 34 are. The purpose of the fences in this gas bladder is to provide connections interiorly of the bladder between its walls to prevent such walls from expanding away from one another under the pressure, i.e., to prevent the gas bladder from “ballooning.” Generally, if a conformal therapy component covers the entire circumference of an area being pressurized, the interior fences are only provided in the liquid bladder. If the component does not cover 360° of the circumference, then the fences (and sometimes some of the dots) can be used to control ballooning of the gas bladder.

In one realization of the preferred embodiment, the liquid was a 20 percent propylene glycol solution in distilled or de-ionized water with a small amount of a wetting agent to break surface tension, and an anti-fungicide such as iodine. The temperature of the liquid was maintained at about 45° F. with a liquid flow rate of about 50-60 lbs./hr. and a liquid pressure of about 10-15 psig. The control unit included an ice bath surrounding a container through which the liquid was circulated after being returned from the heat exchange bladder. The control used in this implementation was capable of supplying liquid at other controlled temperatures. A cyclic air pressure, cycling between 0.25 psig and 1.5 psig every two minutes from peak to peak was introduced into the pressure bladder.

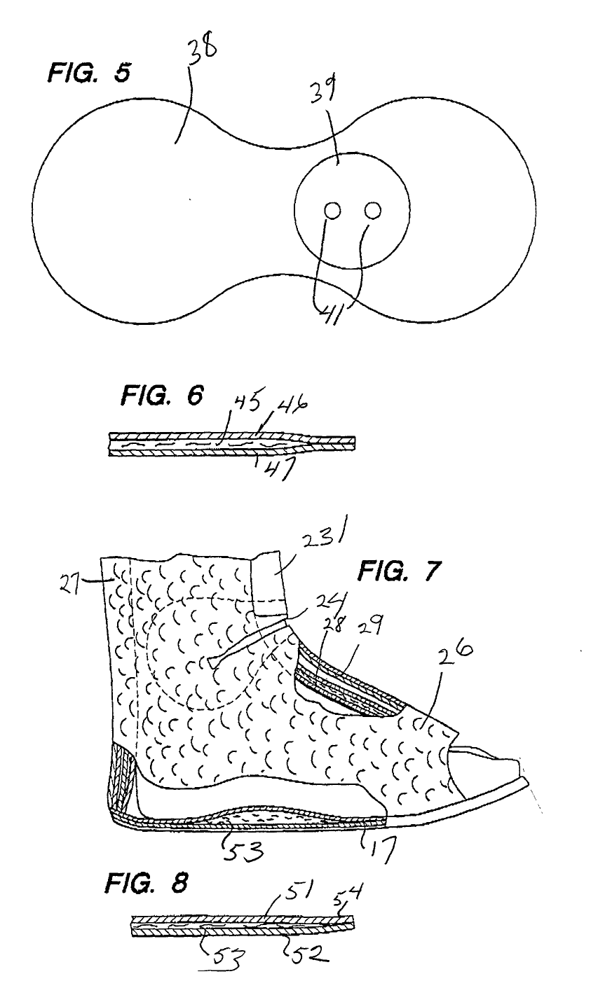

One feature of the invention is that it includes a packet 38 of gel which bridges the gap 24 between the calf 27 and vamp 26 portions of the component. As best illustrated in FIG. 5, the exterior configuration of this packet is like that of a dumbbell or stylized dog bone. It includes a patch 39 adhered to its upper surface, which patch acts to hold a pair of snap fastener halves 41 that are positioned to interact with complementary snap fastener halves indicated at 42 on the covering to hold the packet in place.

Gap 24 must be provided as a practical matter to enable the component to be wrapped about the foot of a wearer. (To make the splint more comfortable for a wearer, the liquid bladder [and the gas bladder] includes edge slits 43 (shown in FIG. 4) covered by an elastic edge binding (not shown).

Gel packet 38 in this embodiment is, in essence, a horizontal tongue. It is used by being placed in the foot/ankle component bridging the gap 24 when this component is wrapped around a foot. In keeping with the invention it contains a thermally conductive gel 45 between its upper and lower walls 46 and 47, respectively. In this connection, such walls are both compliant and liquid impervious. In this preferred embodiment, this thermally conductive gel is formed from the powder Carbopol #672 acrylic homopolymer. This powder is sold by B. F. Goodrich Company of Cleveland, Ohio. Water is mixed with the carbon-based powder according to the instructions. The acrylic polymer of this powder has a slight trace of benzene.

The packet or tongue 38 is positioned at the gap in thermally conductive relationship with the bladder 28 to transfer its thermal energy state to such gap. While in this preferred embodiment the tongue is designed to completely surround the gap, from the broad standpoint this is not necessary. Rather it is only necessary that the packet or tongue be in thermally conductive relationship with the bladder at any location, along, for example, the vamp to transfer its thermal energy state to the part of the foot which is not in thermally conductive relationship with the bladder, i.e., the part at the gap.

As is shown in this preferred embodiment, the gap has a closed periphery and the tongue 38 is engaged with the fluid bladder both above and below the gap 24. The result is that it not only passes on the thermal state on both of the bladder portions, it passes on the pressure. (If such bladder portions were at different temperatures and/or pressures, then the tongue gel would be at an average temperature and pressure.) In fact, in this situation the tongue is in engagement with the temperature controlled, liquid bladder all about the gap.

The invention includes as part of the same, a pressure reactor (reliever) positionable to alleviate the effect of pressure applied by the conformal component to such foot. In this connection, the pressure reactor is most desirably used when a gas pressure bladder as described above is included as part of the combination.

The compliant pressure reactor in this preferred embodiment is the inner gel sole 17 previously mentioned. It is positioned to be at the wearers sole, i.e., at the portion of the foot opposite that at which the gas pressure bladder is provided. As illustrated, it is held in position stretched out along the full sole of the wearer by a pair of hook fastener strips 48 interacting with the previously mentioned loop fastener sole 18.

The gas pressure bladder tends to apply pressure to the foot upper above the wearer's arch and, in essence, tends to “flatten out” such foot. The purpose of the inner gel sole is to resist such pressure and provide support at such arch. This is best seen in FIG. 7. In the preferred embodiment, the gel is also made from the carbon-based acrylic polymer powder sold by B. F. Goodrich with the designation “Carbopol” 672 Polymer. Water is again mixed with the powder according to the instructions.

It should be noted that when it is stated that the pressure reactor is positioned opposite to or opposing the pressure bladder, from a broad standpoint it is meant that it opposes any or all component of the pressure forces on the foot or other body portion which might be unequal and resolves the forces without significant distortion or discomfort. In this situation in which much of the intended applied pressure is 180′ from the pressure reactor, such pressure reactor will tend to be at the same pressure as that of the gas bladder.

FIG. 8 is included to assure there is a complete disclosure. The pressure reactor bladder is made up of a pair of walls 51 and 52 defining a space within which gel 53 is located. The peripheral edges of the walls 51 and 52 are heat sealed together as shown at 54.

As mentioned at the beginning of the detailed description, applicant is not limited to the specific embodiment and variations described above. They are exemplary, rather than exhaustive. The claims, their equivalents and their equivalent language define the scope of protection.

1. A conformal therapy component of an animate body heat exchanger, comprising the combination of:
A. a bladder for confining a heat exchange medium, which bladder is configured to be positioned in thermally conductive relationship with a portion of an animate body; and
B. a packet containing a thermally conductive gel, which packet is positionable to be in thermally conductive relationship with said bladder to transfer a thermal energy state received from said bladder to a part of said animate body not in thermally conductive relationship with said bladder:

2. The therapy component of claim 1 further including a pressure reactor positionable at said portion to alleviate the distortive effect of pressure applied by said component to said portion.

3. The therapy component of claim 1 further including a gas pressure bladder as a part of said combination, said gas pressure bladder configured and positioned to press said heat exchange medium bladder against said portion of said animate body.

4. The therapy component of claim 3 further including a compliant pressure reactor positionable at said portion opposite said gas pressure bladder to alleviate the distortive effect of pressure applied by said gas pressure bladder on said portion.

5. The therapy component of claim 4 for an animate body heat exchanger, which component is designed for the foot/ankle portion of a human and includes vamp and calf parts which are connected together, both said bladder for confining a heat exchange medium and said gas pressure bladder being components of said vamp and calf parts of said component, and said packet being a tongue which bridges a gap defined between said vamp and calf parts at a joint between said parts designed to be at the ankle of a wearer of said component.

6. The therapy component of claim 5 wherein said compliant pressure reactor is an inner sole for said foot/ankle component.

7. The therapy component of claim 5 further including a covering for said vamp and calf parts, which covering has an exterior surface designed to be used as part of a hook and loop fastening arrangement, such surface being one of an exterior loop fastener and hook fastener; and said vamp and calf parts, said vamp and calf parts both including flaps which have surfaces which are the other of said loop and hook fasteners, to interact with the fastener surface on said covering and maintain said vamp and calf parts snugly about the foot/ankle of a wearer.

8. The therapy component of claim 5 wherein when said component is applied to the foot/ankle and calf of a wearer, a gap is defined between said vamp and calf parts, and a plurality of straps are provided for crossing said gap and connecting said vamp and calf parts together at said gap.

9. The therapy component of claim 1 which includes a pair of parts, each of which has a portion of heat exchange medium bladder and which when the component is applied to a portion of an animate body define a gap between such portions, and said packet is positioned to bridge said gap and be in thermally conductive relationship with both portions of said bladder.

10. The therapy component of claim 9 wherein said gap has a closed periphery and said packet is positionable also to be in pressure conductive relationship with said bladder to transfer pressure received therefrom to a portion of said animate body at said gap.

11. The therapy component of claim 10 further including a gas pressure bladder as a part of said combination, said gas pressure bladder configured and positioned to press said heat exchange bladder against said portion of said animate body.

12. A conformal therapy component of an animate body heat exchanger, comprising the combination of:
A. a bladder for confining a heat exchange fluid, said bladder being configured for positioning to apply pressure against said portion of said animate body; and
B. a compliant pressure reactor positionable at said portion opposite said bladder to alleviate the effect of pressure provided by the same on said body portion.

13. The therapy component of claim 12 further including a gas pressure bladder as a part of said combination configured and positioned to press said heat exchange bladder against said portion of said animate body.

14. The therapy component of claim 13 of an animate body heat exchanger, which component is designed for the foot/ankle portion of a human and includes both vamp and calf parts which are connected together with both said bladder for confining a heat exchange fluid and said gas pressure bladder being components of said vamp and calf parts.

15. The therapy component of claim 14 wherein said compliant pressure reactor is an inner sole for said foot/ankle component.

16. The therapy component of claim 14 wherein when said component is applied to the foot/ankle and calf of a wearer, a gap is defined between said vamp and calf parts, and a packet is provided containing a thermally conductive gel positionable to bridge said gap and be in thermally conductive relationship with said bladder for confining a heat exchange fluid, to transfer a thermal energy state received from said bladder to a part of said animate body not in thermally conductive relationship with said bladder.

17. The therapy component of claim 16 wherein said packet is a generally horizontal tongue positionable to bridge said gap when said vamp and calf parts fit about the foot/ankle and calf of a wearer.

18. A conformal therapy component of an animate body heat exchanger comprising the combination of:
A. a bladder for confining a heat exchange fluid, which bladder is configured to be positioned in thermally conductive relationship with a portion of an animate body;
B. a gas pressure bladder for receiving gas pressure, which gas pressure bladder is configured and positioned to press said heat exchange bladder against said portion of the animate body;
C. a compliant pressure reactor positionable at said portion opposite said gas pressure bladder to alleviate the effect of pressure applied by said bladder on said body portion; and
D. A packet containing a thermally conductive gel, which packet is positionable to be in thermally conductive relationship with said bladder for confining a heat exchange fluid, to transfer a thermal energy state received from such bladder to a part of said animate body not in thermally conductive relationship with such bladder for confining a heat exchange fluid.

19. The therapy component of claim 18 of an animate body heat exchanger, which component is designed for the foot/ankle portion of a human and includes both vamp and calf parts which are connected together, said bladder for confining a heat exchange fluid and said gas pressure bladder being components of both said vamp and calf parts of said component, said packet being a generally horizontal tongue which bridges a gap defined to be at the joint between an ankle of a wearer when said component is worn by such wearer.
